(19) United States
(12) Patent Application Publication
(10) Pub. No.: US 2021/0401729 A1
(43) Pub. Date: Dec. 30, 2021

(54) COLLECTIVE LOTION AND SCENT REMOVER

(71) Applicant: Vivere, LLC, MUNCIE, IN (US)

(72) Inventors: Athanasios Chris Karamesines, Muncie, IN (US); Jessica Wang, Chino, CA (US)

(73) Assignee: Vivere, LLC, MUNCIE, IN (US)

(21) Appl. No.: 17/303,526

(22) Filed: Jun. 1, 2021

Related U.S. Application Data
(60) Provisional application No. 63/045,905, filed on Jun. 30, 2020.

(51) Int. Cl. A61K 8/9728; A45D 34/04; A61K 8/02; A61Q 13/00; A61K 8/9794; A61K 8/44; A61K 8/368; A61K 8/60; A61K 8/9789

(52) U.S. Cl. CPC A61K 8/9728; A45D 34/04; A61K 8/0208; A61Q 13/00; A61K 8/9794; A61K 8/44; A61K 2800/88; A61K 8/604; A61K 8/9789; A61K 2800/524; A61K 2800/522; A45D 2200/1036; A45D 2200/25; A61K 8/368

(57) ABSTRACT

A kit for comfortably collecting the scent of one person prolonging it and applying that scent elsewhere on the person or on another person is disclosed. In certain embodiments, the scent collected is the scent from an individual. In still other preferred embodiments, the scent collected is from a sexual partner and is applied elsewhere on that person's body or clothing or to the person's sexual partner. The scent can be collected by applying a collective lotion, which may be provided on a moistened pad, to a user's finger, an applicator or the like and touching or rubbing the desired region. Thereafter, the user's finger or the applicator can be used to apply the scent where desired, such as on their hair, shoulder, arm, chest, external skin area above the upper lip and underneath the nose, hand, wrist, neck, or the like, or on those areas of another desired person. A scent removed may also be provided to subsequently remove the scent transferred as desired.

### CROSS REFERENCE TO RELATED APPLICATION

This application claims the benefit of U.S. Provisional Application No. 63/045,905 filed Jun. 30, 2020 of which is hereby incorporated by reference.

### BACKGROUND

The olfactory system and associated organs is used for the sense of smell (olfaction) in mammals. The sense of smell is part of the human chemosensory system and allows humans to sense a wide variety of chemicals, including potentially sex pheromones or the like.

The limbic system is the portion of our brain that controls emotion, aids in memory retention, and contains one of our most prominent pleasure centers. It produces desire, arousal and sensuous activity. Compared to our other senses, smell is unique. It is the only one of the five senses able to bypass sensory parts of the brain that relay and interpret information and can trigger an immediate response.

When a human inhales a pleasurable or familiar scent, it can elicit vivid emotions and sensual stimulation. Scent-invoked memories accelerate these features as they are processed through the brain. Our olfactory system can transport us through time, such as scents experienced from days-gone-by, the scent of a personal item from a departed love, a local restaurant, or a bakery. Scent produces a greater emotional impact than remembrances sparked by sights, sounds, or other sensory cues. The human body contains only two receptors for sight, four for touch, but over three-hundred for the sense of smell.

As such, as need exists for less-invasive products and a method that provides one with the ability to comfortably collect and create lasting individual scent(s).

### SUMMARY

In one embodiment, a kit for comfortably collecting and prolonging the scent of one person, and applying the scent of that person elsewhere on themselves or on another person is disclosed. The kit may include a scent remover for later removing the scent when desired, or as a gentle fragrance free method that enables one to cleanse prior to application of the collective lotion.

In certain embodiments, the scent collected is the scent from an individual. In still other preferred embodiments, the scent collected is from a sexual partner and is applied elsewhere on that person's body or clothing or to the person's sexual partner. In one preferred embodiment, the scent collected is from the neck, chest, underarm, genital area or reproductive organ such as the vagina or penis. It has been found that for women vaginal copulins can be collected and applied elsewhere on the woman or her partner to enhance sexual experience for one or both parties. With respect to a man, odoriferous sweat (such as that produced by the apocrine glands) may be collected from the man's chest, underarm or external genital area and applied elsewhere on the man or his partner to similar enhance the sexual experience for one or both parties.

The scent can be collected by applying a collective lotion, which may be provided on a moistened pad, to a user's finger or the like and suitably touch or massage the desired skin area to collect an individual scent. Thereafter, the user's finger can be used to apply the scent where desired, such as on their hair, shoulder, arm, chest, external skin area above the upper lip and underneath the nose, hand, wrist, neck, or the like, or on those areas of another desired person.

In a preferred embodiment, kits of the present disclosure comprise a collective lotion pad and an scent removing towelette. In additional aspects, the pad and towelette of disclosed kits may be sealed in packaging. In other aspects, the packaging may comprise two discrete compartments so that each of the pad and towelette are segregated from the other. In a preferred embodiment, a collective pad or wipe comprises a collective lotion applied thereon. In an additional preferred embodiment, a scent removal towelette or wipe comprises a composition to remove and/or neutralize scents, smells or odors.

In some embodiments, the packaging sealing kits of the present disclosure may comprise aluminum, polymers, and/or combinations of aluminum and polymers. For example, in one preferred embodiment, the packaging of the disclosed kits comprises layers of polyethylene terephthalate, aluminum, and/or one or more linear low density polyethyelene polymers. In a preferred embodiment, the packaging of the disclosed kits comprises a material having layers of PET12, AL6, and/or LLDPE60.

In one embodiment, a lotion, such as a collective lotion may comprise one or more oils, such as a vegetable oil, including but not limited to sunflower oil, almond oil; one or more vitamins, including but not limited to Vitamin E; cocoa butter; beeswax; one or more fragrances; and/or one or more dyes. In a preferred embodiment, a lotion, such as a collective lotion may be applied to a wipe, pad, towelette, flushable wipe, and/or cloth.

In one embodiment, an scent removing composition may comprise water; aloe vera; panthenol; one or more preservatives, such as for example but not limited to potassium sorbate and/or sodium benzoate; an alkyl polyglucoside, such as for example but not limited to, Plantaren 2000 N UP; a Saccharomyces Ferment; one or more humectants, such as for example including but not limited to Zeema propanediol; and/or one or more extracts, such as for example but not limited to cucumber extract and chamomile extract. In a preferred embodiment, such a scent removing composition may be applied to a wipe, pad, towlette, flushable wipe, and/or cloth.

Additional embodiments of the invention, as well as features and advantages thereof, will be apparent from the descriptions herein.

### BRIEF DESCRIPTION OF THE DRAWINGS

FIG. 1 shows a digital image of a kit of the present disclosure including a collective lotion pad and a scent remover towelette, each packaged in separate discrete packaging.

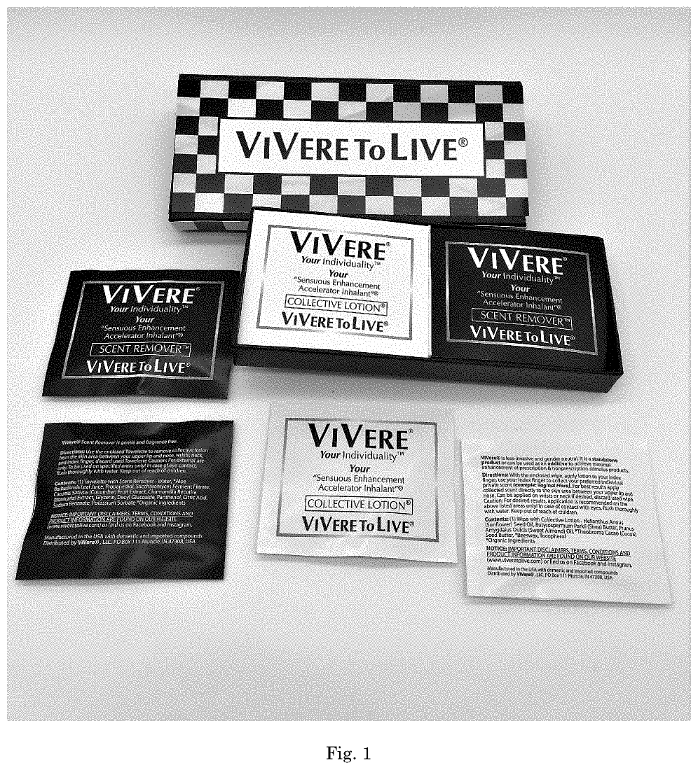

FIG. 2 show a digital image of a kit of the present disclosure including a collective lotion pad and a scent remover towelette, each packaged in separate discrete packaging, opened here with each partially remove from the packaging.

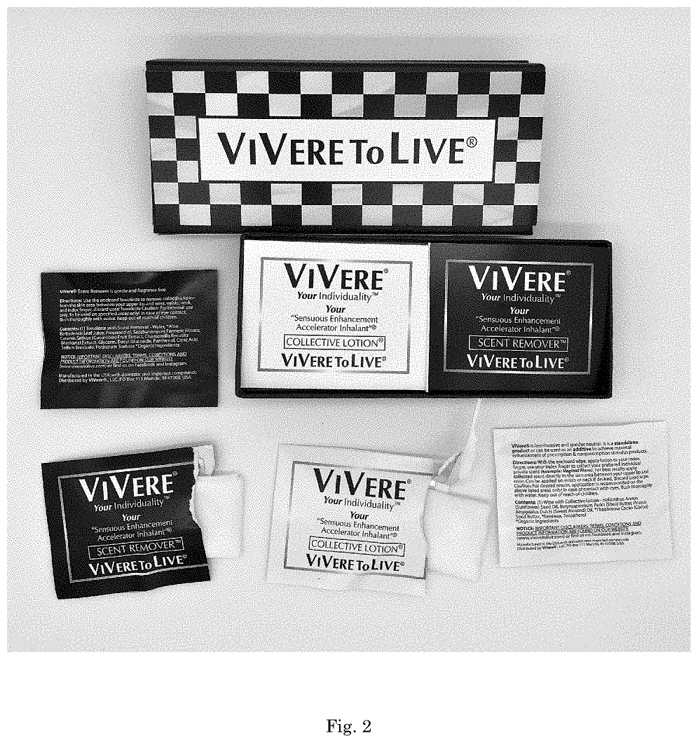

### DETAILED DESCRIPTION

For the purposes of promoting an understanding of the principles of the invention, reference will now be made to certain embodiments and specific language will be used to describe the same. It will nevertheless be understood that no limitation of the scope of the invention is thereby intended, such alterations and further modifications, and such further applications of the principles of the invention as described herein being contemplated as would normally occur to one skilled in the art to which the invention relates. Additionally, in the detailed description below, numerous alternatives are given for various features. It will be understood that each such disclosed alternative, or combinations of such alternatives, can be combined with the more generalized features discussed in the Summary above, or set forth in the embodiments described below to provide additional disclosed embodiments herein.

In one embodiment, a kit for comfortably collecting the scent of one person, prolonging it and applying that scent in another place on the person from which it was collected or on another person is disclosed. The kit may include a scent remover for later removing the scent when desired, or as a gentle fragrance free method that enables one to cleanse prior to application of the collective lotion.

In certain embodiments, the scent collected is the scent from an individual. In still other preferred embodiments, the scent collected is from a sexual partner and is applied elsewhere on that person's body or clothing or to the person's sexual partner. In one preferred embodiment, the scent collected is from the neck, chest, underarm, genital area or reproductive organ such as the vagina or penis. It has been found that for women vaginal copulins can be collected and applied elsewhere on the woman or her partner to enhance sexual experience for one or both parties. With respect to a man, odoriferous sweat may be collected from the man's chest, underarm area or external genital area and applied elsewhere on the man or his partner to similarly enhance the sexual experience for one or both parties.

The scent can be collected by applying a collective lotion, which may be provided on a moistened pad, to a user's finger, an applicator or the like and suitably touch or massage the desired skin area to collect an individual scent. Thereafter, the user's finger, the applicator or the like can be used to apply the scent where desired, such as on their hair, shoulder, arm, chest, external skin area above the upper lip and underneath the nose, hand, wrist, neck, or the like, or on those areas of another desired person.

In a preferred embodiment, kits of the present disclosure comprise a collective pad and an scent removing towelette. In additional aspects, the pad and towelette of the disclosed kit may be sealed in packaging. In other aspects, the packaging may comprise two discrete compartments or two separate packages so that the pad and towlette are segregated from each other. In a preferred embodiment, a collective pad comprises a collective lotion applied thereon. In an additional preferred embodiment, a scent removing wipe comprises a composition to remove and/or neutralize scents and/or smells.

In some embodiments, the packaging sealing kits of the present disclosure may comprise aluminum, polymers, and/or combinations of aluminum and polymers. For example, in one preferred embodiment, the packaging of the disclosed kits comprises layers of polyethylene terephthalate, aluminum, and/or one or more linear low density polyethyelene polymers. In a preferred embodiment, the packaging of the disclosed kits comprises a material having layers of PET12, AL6, and/or LLDPE60.

In one embodiment, a lotion, such as a collective lotion may comprise one or more oils, such as a vegetable oil, including but not limited to sunflower oil, almond oil; one or more vitamins, including but not limited to Vitamin E; cocoa butter; beeswax; one or more fragrances; and/or one or more dyes. In a preferred embodiment, a lotion, such as a collective lotion may be applied to a wipe, pad, towlette, flushable wipe, and/or cloth. The collective lotion is gender neutral. It is a stand-alone product, but was also developed with qualities to be used as an additive to achieve maximal benefits of prescription and non-prescription stimulus products.

In one embodiment, a scent removing composition may comprise water; aloe vera; panthenol; one or more preservatives, such as for example but not limited to potassium sorbate and/or sodium benzoate; an alkyl polyglucoside, such as for example but not limited to, Plantaren 2000 N UP; a Saccharomyces Ferment; one or more humectants, such as for example including but not limited to Zeema propanediol; and/or one or more extracts, such as for example but not limited to cucumber extract and chamomile extract. In a preferred embodiment, such a scent removing composition may be applied to a towlette, flushable wipe, cloth or the like.

In order to promote a further understanding of the present invention and its various embodiments, the following specific examples are provided. It will be understood that these examples are illustrative and not limiting of the invention.

### Example 1

### Preparation or Manufacture of Collective Lotion Pad

A collective lotion pad was prepared by first mixing the collective lotion. To a cleaned vessel is added about 97% sunflower oil, about 1% Lipex 205 deodorized vegetable oil, about 0.5% almond oil, about 0.3% Vitamin E, about 0.2% organic cocoa butter, and about 0.2% organic beeswax with hominization by a propeller on medium speed.

Once prepared, the collective lotion was applied to a pad, or towelette by procedures known to those of ordinary skill in the art, for example by coating the collective lotion on a substrate, and then cutting and folding the substrate into the form of a pad or towelette.

### Example 2

### Preparation or Manufacture of Scent Remover Towelette

A scent remover towelette was prepared by first mixing the scent remover composition. To a cleaned vessel is added about 90% deionized water, about 0.1% aloe vera, about 0.05% DL panthenol, about 0.25% potassium sorbate, about 0.45% sodium benzoate, about 0.2% Plantaren 2000 N UP diol, about 3% DeoPlex M0953 Saccharomyces Ferment, about 0.1% Cucumber Extract in glycerin, and about 0.1% Chamomile Extract in glycerin.

Once prepared, the scent remover composition was applied to a towelette, or pad by procedures known to those of ordinary skill in the art, for example by coating or spraying the scent remover composition on a substrate, and then cutting and folding the substrate into the form of a towelette or pad.

The uses of the terms “a” and “an” and “the” and similar references in the context of describing the invention (especially in the context of the following claims) are to be construed to cover both the singular and the plural unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the drawings and the foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

1-23. (canceled)

24. A composition of matter for the removal of the scent of another comprising:
a skin conditioner;
a surfactant;
a humectant;
an odor neutralizer;
a preservative; and
water.

25. The composition of matter of claim 24, wherein the conditioner comprises Aloe Vera Extract, Panthenol, Cucumber Extract, or Chamomile Extract.

26. The composition of matter of claim 24, wherein the surfactant comprises Decyl Glucoside.

27. The composition of matter of claim 24, wherein the humectant comprises propanediol.

28. The composition of matter of claim 24, wherein the odor neutralizer comprises Saccharomeyes Ferment.

29. The composition of matter of claim 24, wherein the preservative comprises Potassium Sorbate or Sodium Benzoate.

30. The composition of matter of claim 24, further comprising citric acid.

31. The composition of matter of claim 24, wherein the water comprises about 85% by weight to about 95% by weight of the composition.

32. (canceled)

33. The composition of matter of claim 24, wherein the skin conditioner comprises about 0.01% by weight to about 5% by weight of the composition.

34-40. (canceled)

41. The composition of matter of claim 24, wherein the odor neutralizer comprises between about 1% by weight and about 10% by weight of the composition.

42. The composition of matter of claim 24, wherein the odor neutralizer comprises between about 2% by weight and about 9% by weight of the composition.

43. The composition of matter of claim 24, wherein the odor neutralizer comprises between about 3% by weight and about 8% by weight of the composition.

44. The composition of matter of claim 24, wherein the odor neutralizer comprises between about 4% by weight and about 7% by weight of the composition.

45. The composition of matter of claim 24, wherein the odor neutralizer comprises between about 5% by weight and about 6% by weight of the composition.

46. The composition of matter of claim 24, wherein the odor neutralizer comprises between about 5% by weight of the composition.

47. A composition of matter comprising the compositions of claim 24 and a substrate.

48. The composition of matter of claim 46, wherein the substrate comprises a wipe or towellette.

49-50. (canceled)

51. A method of using the kit of claim 46 comprising the acts of:
collecting the scent of a person using the composition of claim 24;
applying the scent collected using the composition of claim 24 to a different location on the person or on a second person.

52-53. (canceled)

54. The method of claim 50, wherein the method increases the sexual enjoyment of a sexual partner.
